(19) United States
(12) Patent Application Publication
(10) Pub. No.: US 2025/0352754 A1
(43) Pub. Date: Nov. 20, 2025

(54) ARRANGEMENT WITH A RESPIRATORY MASK AND AN INTERMEDIATE PIECE, AND METHODS FOR OPERATING SUCH AN ARRANGEMENT

(71) Applicant: Dräger Safety AG & Co. KGaA, Lübeck (DE)

(72) Inventors: Matti Köhn, Lübeck (DE); Niels Milde, Lübeck (DE)

(21) Appl. No.: 19/208,301

(22) Filed: May 14, 2025

(30) Foreign Application Priority Data
May 15, 2024 (DE) 102024113535.2

(51) Int. Cl. A61M 16/08; A61M 16/00; A61M 16/06; A61M 16/20

(52) U.S. Cl. CPC A61M 16/0858; A61M 16/06; A61M 16/209; A61M 2016/0027; A61M 2205/583

(57) ABSTRACT

The disclosure relates to an arrangement with a breathing air source, a respiratory mask, and an intermediate piece, as well as to two methods for operating such an arrangement. A device-side coupling point of the intermediate piece can be connected to a coupling point of the breathing air source, a mask-side coupling point of the intermediate piece can be connected to a coupling point of the respiratory mask. If the intermediate piece is connected to the breathing air source (30) and to the respiratory mask, a fluid connection is established which leads from the breathing air source to the respiratory mask. A pressure sensor measures the pressure inside the intermediate piece. In addition, the breathing air source can be connected directly to the respiratory mask.

### CROSS-REFERENCE TO RELATED APPLICATIONS

This application claims the priority of German Application No. 102024113535.2, filed on May 15, 2024, the entire contents of which being fully incorporated herein by reference.

### DESCRIPTION

The disclosure relates to an arrangement with a respiratory mask and an intermediate piece, and a plurality of methods for operating such an arrangement.

A user may place a respiratory mask on his or her face while the user is in an environment where particulate matter and other contaminants are or may be present. The respiratory mask can be connected to a filter unit or to a compressed air breathing apparatus, and filtered or clean breathing air reaches the user through this fluid connection. The respiratory mask prevents pollutants from bypassing the filter unit and reaching the user.

In order for the respiratory mask to achieve the desired protective effect, it must fit fluid-tight to the user's face. DE 10 2022 123 988 B3 describes a device that allows a user to check the tightness of a respiratory mask.

The disclosure is based on the object of providing an arrangement comprising a respiratory mask, which arrangement should make it possible to check the respiratory mask for leaks better than known arrangements. Furthermore, the disclosure is based on the object of providing methods for operating such an arrangement.

The object is achieved by an arrangement having the features of claim 1 and by two methods having the features of claims 6 and 8. Additional embodiments are specified in the dependent claims. Additional embodiments of the arrangement according to the disclosure are, as far as appropriate, also additional embodiments of at least one method according to the disclosure, and vice versa.

Note: The order in which a method claim lists steps of a claimed method does not necessarily specify a temporal order in which those steps are performed.

The arrangement according to the disclosure comprises a respiratory mask. The respiratory mask is configured to fit snugly (rest) against the face of a user, ideally in a fluid-tight manner. The arrangement also includes a breathing air source. The breathing air source is configured for providing breathing air or another breathable gas mixture. A human can inhale this breathable gas mixture.

The arrangement further comprises an intermediate piece (intermediate part) with a coupling point on the device side and a coupling point on the mask side. The mask-side coupling point of the intermediate piece can be detachably connected to a corresponding coupling point of the respiratory mask. The device-side coupling point of the intermediate piece can be detachably connected to a corresponding coupling point of the breathing air source. The intermediate piece provides a fluid connection between the two coupling points of the intermediate piece.

As already mentioned, the device-side coupling point of the intermediate piece can be connected to the coupling point of the breathing air source. The mask-side coupling point of the intermediate piece can be connected to the coupling point of the respiratory mask. If these two connections are established, the following situation occurs: At least temporarily, a first fluid connection is established or can be established, this first fluid connection leading from the breathing air source to the respiratory mask. This first fluid connection passes through the intermediate piece. The fluid connection provided by the intermediate piece is therefore a part of the first fluid connection from the breathing air source to the respiratory mask.

In addition, the coupling point of the breathing air source can be detachably connected to the coupling point of the respiratory mask, i.e. without the intermediate piece being located between the breathing air source and the respiratory mask. If these two coupling points are connected to each other, a second fluid connection is established, this fluid connection leading from the breathing air source to the respiratory mask. This second fluid connection is established directly, i.e. without the use of the intermediate piece. The second fluid connection bypasses the intermediate piece.

A pressure sensor in the arrangement is configured to measure the pressure at a measuring position. This measuring position is located in the fluid connection which the intermediate piece provides, and which interconnects the two coupling points of the intermediate piece. The measuring position is therefore located between the two coupling points of the intermediate piece. The pressure sensor measures the pressure in the interior of the intermediate piece. Preferably, the pressure sensor is mechanically connected to the intermediate piece.

Note: The wording is used that a sensor is configured to measure a physical quantity, for example a pressure. This wording means that the sensor is configured to directly measure the physical quantity or at least one other quantity that correlates with the quantity to be measured. The or one other measured quantity or the combination of the other measured quantities together are therefore an indicator for the physical quantity to be measured. The measurement provides at least one value for the physical variable sought.

In particular, the arrangement according to the disclosure can be operated in the following different modes:
The intermediate piece is connected both to the breathing air source and to the respiratory mask, and at least temporarily a fluid connection (the first fluid connection) is established from the breathing air source through the intermediate piece to the respiratory mask.
The breathing air source is connected directly to the respiratory mask, so that a fluid connection (the second fluid connection) is established from the breathing air source to the respiratory mask. The intermediate piece is neither connected to the breathing air source nor to the respiratory mask.
The intermediate piece is connected to the respiratory mask but not to the breathing air source, so that a fluid connection is established from the environment through the intermediate piece to the respiratory mask.
The intermediate piece is connected to the breathing air source, but not to the respiratory mask. Preferably, the mask-side coupling point of the intermediate piece is closed with a closure.
The intermediate piece is neither connected to the breathing air source nor to the respiratory mask. The breathing air source is not connected to the respiratory mask.

In the first mode (intermediate piece connected to the breathing air source and to the respiratory mask), the following steps can be carried out, especially while the respiratory mask is in contact with a user's face:

It is possible to determine the pressure of the breathing air in an interior space between the respiratory mask and the user's face, this breathing air originating from the breathing air source.

The breathing air source provides breathing air, and there is a specified target pressure, at which target pressure the breathing air source should provide the breathing air. The breathing air source includes, for example, a compressed air breathing apparatus. The pressure measured at the measuring position by the pressure sensor of the arrangement is compared with the specified target pressure. A large deviation between the target pressure and the pressure measured by the pressure sensor at the measuring position may be an indication that a leak has occurred, that the respiratory mask is not resting against the user's face in a fluid-tight manner, or that the breathing air source is not operating properly.

In the second mode (breathing air source directly connected to the respiratory mask), the arrangement can be used productively without the intermediate piece hindering use. The intermediate piece can be cleaned or visually inspected.

In the third mode (intermediate piece connected to the respiratory mask but not to the breathing air source), a user can inhale air from the environment and check whether the respiratory mask fits the face in a fluid-tight manner. A fluid connection is established between the environment and the respiratory mask, which connection passes through the intermediate piece. Preferably, the intermediate piece comprises a valve, the valve selectively closing or releasing the fluid connection depending on the valve's position. The valve opens if the user inhales strongly enough, i.e. if the pressure in the space between the valve and the respiratory mask is lower than the ambient pressure by a predetermined pressure threshold. Instead of a valve, a closure can also be used, it is possible for the closure to be placed on the intermediate piece and then interrupting (blocking) the fluid connection between the respiratory mask and the environment.

In the fourth mode (intermediate piece connected to the breathing air source, mask-side coupling point closed by a closure), the pressure that the breathing air source actually provides can be measured. This measurement is not influenced by the respiratory mask.

In the fifth mode (intermediate piece not connected to the breathing air source or the respiratory mask, and the respiratory mask not connected to the breathing air source), the respiratory mask, the breathing air source, and the intermediate piece can be cleaned or visually inspected.

The intermediate piece may comprise a valve. The valve can be switched to a closing state and to a releasing state. In the closing state, the valve blocks the fluid connection which connects the two coupling points of the intermediate piece, and which is provided by the intermediate piece. In the releasing state, the valve releases this fluid connection. The valve is in the releasing state if the following situation occurs: The pressure at the mask-side coupling point is lower than the pressure at the device-side coupling point by at least a specified pressure difference. Therefore, a greater pressure at the device-side coupling point than at the mask-side coupling point occurs. In this situation, there is a pressure difference between the two sides of the valve, the pressure difference being at least as large as the specified pressure difference. If this situation does not occur, the valve is in the closing state. The valve can be implemented as a purely mechanical component and, in particular, does not require any electrical or hydraulic or pneumatic energy.

The embodiment with the valve makes it possible to check whether or not the respiratory mask fits fluid-tight to the face of a user. In one application, the user creates a negative pressure at the mask-side coupling point by inhaling. If the user breathes in (inhales) strongly enough, the valve opens. In another application, the breathing air source generates an overpressure at the device-side coupling point, which overpressure allows the breathing air source to be checked. A sufficiently large overpressure should open the valve. If the valve does not open, the breathing air source is not able to generate sufficient overpressure, which is usually a fault.

According to the disclosure, the arrangement comprises a pressure sensor, and the pressure sensor is configured to measure the pressure at a measuring position in the fluid connection. This fluid connection is provided by the intermediate piece. The measured pressure occurs in the interior of the intermediate piece. In one embodiment, the pressure sensor comprises a display unit. The display unit can show a measured value for the pressure. This display unit may be positioned as follows: If the mask-side coupling point of the intermediate piece is connected to the coupling point of the respiratory mask, the display unit points towards the respiratory mask. A user of the respiratory mask can read the display unit and thus determine for himself/herself what pressure is currently occurring in the fluid connection. If the intermediate piece is connected to a breathing air source, this configuration allows a user to read a measured pressure in the breathing air source displayed on the display unit.

Preferably, the display unit shows the pressure relative to the ambient pressure. The display unit can therefore show a positive or a negative pressure.

According to the disclosure, the arrangement comprises a breathing air source, an intermediate piece, and a pressure sensor. In one embodiment, the arrangement additionally comprises an additional (a further) breathing air source with a coupling point, an additional intermediate piece, and an additional pressure sensor. The additional breathing air source can also provide breathing air. Just like the intermediate piece, the additional intermediate piece also includes a device-side coupling point and a mask-side coupling point. The additional intermediate piece also establishes a fluid connection between its two coupling points. The additional pressure sensor is configured to measure the pressure at an additional measuring position, this additional measuring position being located in the fluid connection provided by the additional intermediate piece. Therefore, the additional pressure sensor measures the pressure in the interior of the additional intermediate piece. Preferably, the additional pressure sensor is mechanically connected to the additional intermediate piece.

The device-side coupling point of the additional intermediate piece can be detachably connected to a corresponding coupling point of the additional breathing air source. Therefore, the coupling point of the respiratory mask can selectively be connected either to the mask-side coupling point of the intermediate piece or to the mask-side coupling point of the additional intermediate piece. If the additional intermediate piece is connected to the respiratory mask and to the additional breathing air source, a fluid connection between the additional breathing air source and the respiratory mask is established (the additional first fluid connection). This fluid connection guides through the additional intermediate piece, therefore comprises the fluid connection which is provided by the additional intermediate piece.

The embodiment with the additional breathing air source and the additional intermediate piece makes it possible to test the same respiratory mask both in conjunction with the breathing air source and in conjunction with the additional breathing air source. While this test is being performed, the user can keep the respiratory mask on his face. This embodiment saves time when checking the two breathing air sources.

In one embodiment, the coupling point of the additional breathing air source can also be connected to the coupling point of the respiratory mask, the additional intermediate piece not being required for this connection. In particular, this embodiment makes it possible, to replace a used breathing air source with a new breathing air source without the user having to remove (take off) the respiratory mask.

A preferred method for operating an arrangement according to the disclosure comprises the following steps:
The respiratory mask is placed on the face of a user.
The intermediate piece is connected to the respiratory mask and to the breathing air source. For this purpose, the corresponding coupling points are connected to each other. After this connection is established, at least temporarily the first fluid connection is established or can be established, this first fluid connection guiding air from the breathing air source through the intermediate piece to the respiratory mask.
The pressure sensor measures the pressure at the measuring position in the intermediate piece at least once while the intermediate piece is connected to the breathing air source and to the respiratory mask. As a rule, the pressure at the measuring position is mainly caused by the breathing air source.
Afterwards, the intermediate piece is separated from the respiratory mask and from the breathing air source.
The breathing air source is connected directly to the respiratory mask. This establishes the second fluid connection that leads from the breathing air source to the respiratory mask. The intermediate piece is not used.

The breathing air source and the respiratory mask can now be used for productive purposes without using the intermediate piece and without the intermediate piece hindering this use.

According to the method just described, the intermediate piece is connected both to the breathing air source and to the respiratory mask. In one embodiment, the intermediate piece is first connected to the breathing air source, and then the intermediate piece is connected to the respiratory mask, preferably in a state in which the intermediate piece is already connected to the breathing air source.

After the intermediate piece is connected to the breathing air source and before the intermediate piece is connected to the respirator, preferably the following steps are carried out:
The mask-side coupling point of the intermediate piece is sealed in a fluid-tight manner by means of a closure.
The pressure sensor measures the pressure at the measuring position at least once while the mask-side coupling point is closed in a fluid-tight manner. Again, the pressure at the measuring position is essentially generated by the breathing air source.
Afterwards, the closure is removed.
Afterwards, the mask-side coupling point is connected to the coupling point of the respiratory mask.

The breathing air source and the respiratory mask can now be used for productive purposes.

This embodiment makes it possible to measure the pressure that the breathing air source currently can provide. It is possible to operate the breathing air source in different modes and to measure the respectively provided pressure in each mode.

A further method specifies a method for operating an arrangement according to an embodiment of the disclosure. The method relates to an arrangement comprising a valve in the intermediate piece, as just described.

The method includes the following steps:
The respiratory mask is placed on the face of a user.
The intermediate piece is or remains connected to the respiratory mask.
The intermediate piece is or remains separated from the breathing air source. As a result, at least temporarily, a fluid connection from an environment of the arrangement to the respiratory mask is established or can be established. This fluid connection passes through the intermediate piece.
The valve is moved into or kept in the closing position. This movement interrupts the fluid connection with the environment.
The pressure sensor measures the pressure at the measuring position at least once while the valve is in the closing position. In particular, the measured pressure can be used to check whether the respiratory mask is actually fluid-tight with the user's face.
Afterwards, the intermediate piece is separated from the respiratory mask.
The breathing air source is connected to the respiratory mask. More specifically: The coupling point of the breathing air source is connected to the coupling point of the respiratory mask. This establishes the second fluid connection that leads from the breathing air source to the respiratory mask.

The breathing air source and the respiratory mask can now be used.

In the following, the disclosure is described on the basis of an exemplary embodiment. In the drawings,

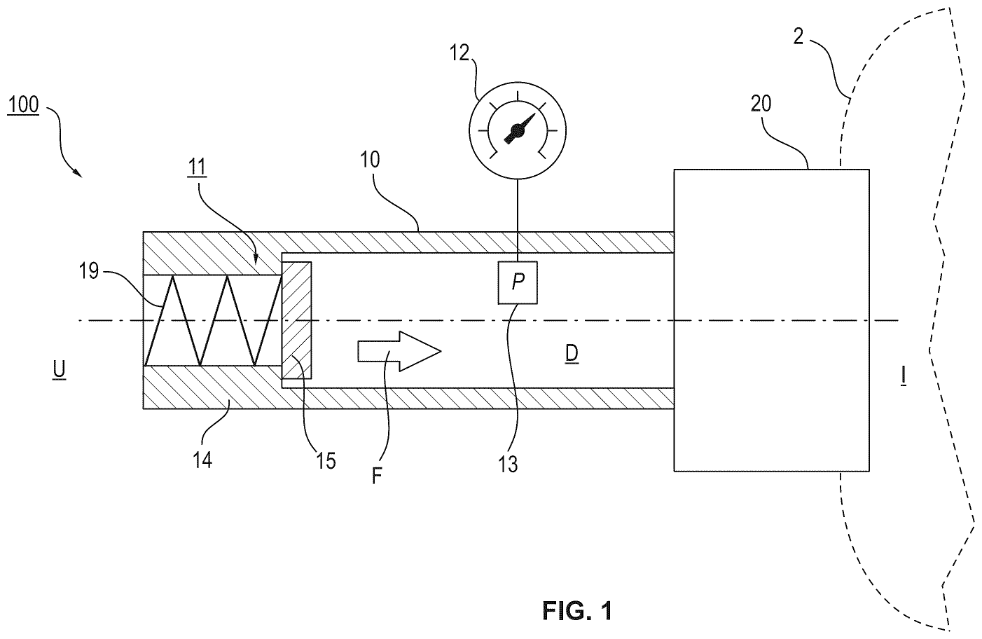

FIG. 1 shows in a schematic view a first embodiment of an intermediate piece;

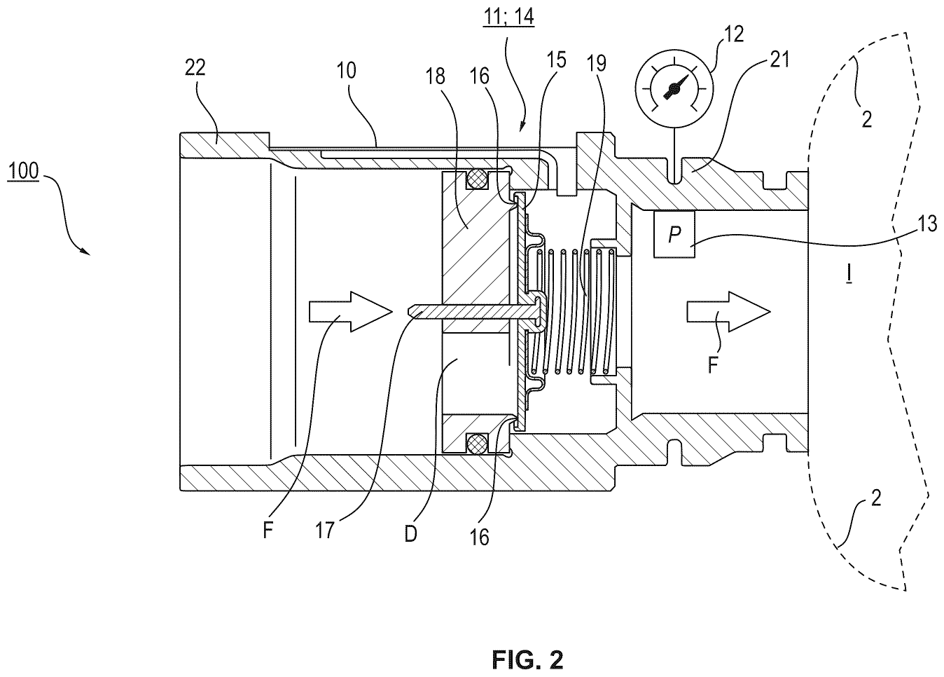

FIG. 2 shows in a cross-sectional view a second embodiment of the intermediate piece;

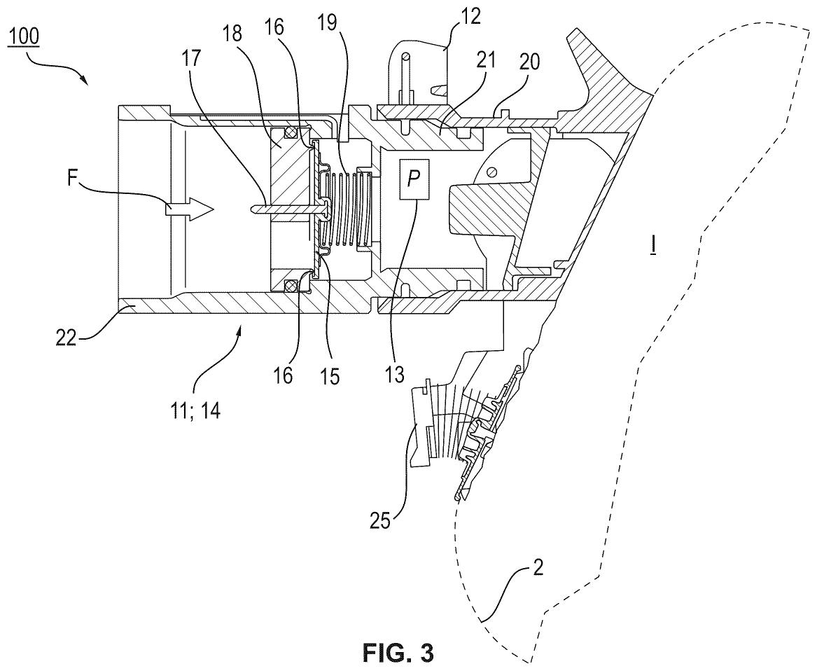

FIG. 3 shows in a cross-sectional view a third embodiment of the intermediate piece;

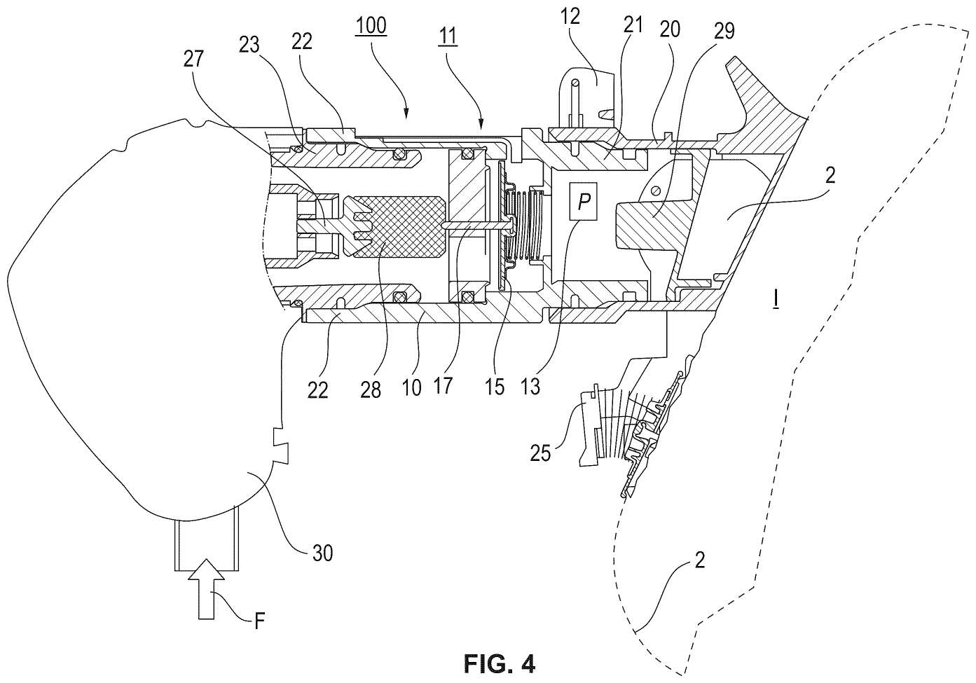

FIG. 4 shows the intermediate piece of FIG. 3 between a respiratory mask and a lung demand valve;

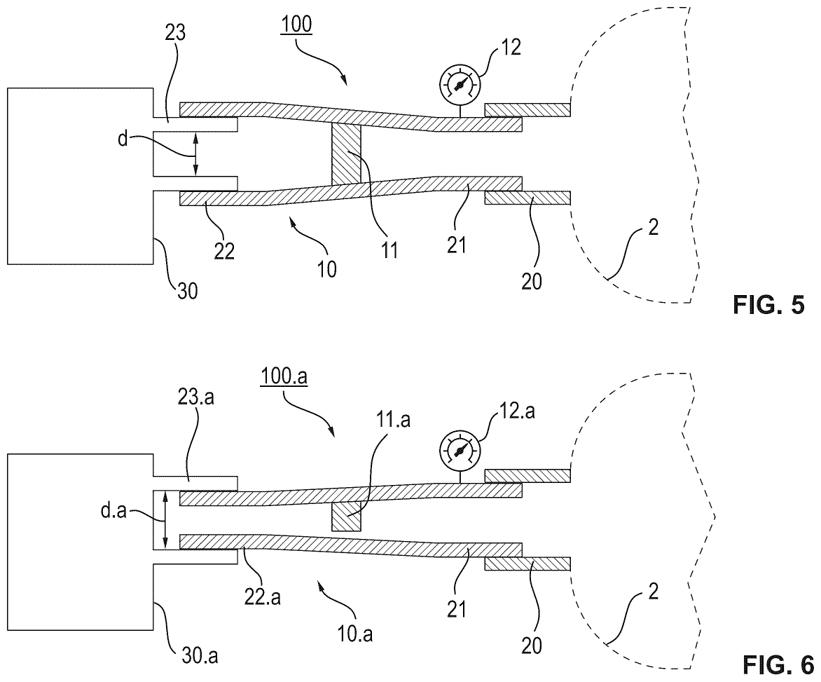

FIG. 5 shows an arrangement with a larger coupling point of the breathing air source and a corresponding intermediate piece;

FIG. 6 shows an arrangement with a smaller coupling point of the breathing air source and a corresponding intermediate piece.

FIG. 1 shows a piece 100 which is known from DE 10 2022 123 988 B3. Such a piece can also be used as part of the arrangement according to the disclosure and can function as the intermediate piece.

A respiratory mask 2, shown only schematically, rests on the face of a user, ideally in a fluid-tight manner. An interior space I is formed between the user's face and the respiratory mask 2. A base body 10 of the piece 100 can be inserted into a connection opening of a coupling point 20 of the respiratory mask 2. The base body 10 comprises a tubular fluid guide unit which can provide a fluid connection D. The inserted base body 10 establishes the fluid connection D between the interior space I and an environment U, this fluid connection D leading through the base body 10.

A valve 11 is arranged in the fluid connection D at that end of the base body 10 which points away from the respiratory mask 2. The valve 11 comprises a valve body 15 and a mechanical spring 19. In the embodiment of FIG. 1, the spring 19 is a tension spring. The spring 19 strives to pull the valve body 15 against a valve body seat 14 with a sealing surface 16, see FIG. 2. FIG. 1 shows the valve 11 in a closed position in which the valve body 15 rests against the valve body seat 14 and the valve 11 interrupts the fluid connection D.

A sufficiently large overpressure in the environment U relative to the interior space I causes the valve body 15 to be moved away from the valve body seat 14 against the force of the spring 19, thereby causing a gap to appear between the valve body 15 and the valve body seat 14. The movement moves the valve 11 into a releasing position. If the valve 11 is in the releasing position, the fluid connection D between the environment U and the interior space I is established. The valve 11 opens if the pressure in the environment U is greater than the pressure in the interior space I by a predetermined limit (threshold)—or, in other words, if there is a sufficiently large negative pressure in the interior space I. In particular, the negative pressure at which the valve 11 is transferred to the released position depends on the embodiment of the spring 19, especially on the spring constant and the position at which the spring 19 is supported. In one embodiment, the valve 11 opens at a negative pressure between 14 mbar and 15 mbar.

A pressure gage (pressure gauge, manometer) 12 is arranged on the base body 10. The pressure gage 12 measures the pressure at a measuring position 13. This measuring position 13 is located inside the base body 10 and between the valve 11 and a mask-side coupling point 21 of the base body 10, the mask-side coupling point 21 being described below. The measuring position 13 is therefore located in the fluid connection D. The mask-side coupling point 21 is connected to the coupling point 20. The pressure gage 12 comprises a display surface on which the measured pressure is shown. The pressure gage 12 shows the measured pressure on this display surface. The pressure gage 12 may indicate the difference between the pressure in the interior space I and the pressure in the environment U. This display surface may be—in contrast to the illustration in FIG. 1—positioned such that a user of the respiratory mask 2 can read the pressure displayed on the display surface without having to remove (take-off) the respiratory mask 2 or to disconnect the piece 100.

The piece 100 includes the base body 10, the valve 11, and the pressure gage 12.

One possible application of piece 100 is the following: A user places the respiratory mask 2 on his/her face and checks whether the respiratory mask 2 actually fits fluid-tight to the face and thereby prevents particles and other pollutants occurring in the environment U from entering the interior space I. For this leak test, the user breathes in (inhales). In this way, the user creates a negative pressure in the interior space I. Initially, the valve 11 is closed. As soon as the user generates a sufficiently large negative pressure by inhaling, the valve 11 opens, and the user can inhale breathing air from the environment U. The pressure gage 12 measures the pressure in the fluid guide unit D, more precisely at the measuring position 13, preferably as a negative pressure relative to the ambient pressure. The pressure gage 12 visually displays the measured pressure. The user can read off the pressure gage 12 without removing the respiratory mask 2. This allows the user to determine whether and, if so, at what pressure, the valve 11 opens. If the respiratory mask 2 does not fit fluid-tight against the user's face, a lower negative pressure (smaller pressure difference) occurs in the fluid connection D until the valve 11 opens, compared to a fluid-tight respiratory mask 2.

FIG. 2 shows a cross-sectional view of a second embodiment of the piece 100, FIG. 3 shows a third embodiment. The same reference signs have the same meanings as in FIG. 1.

In this embodiment, the spring 19 is a compression spring (pressure spring) which strives to press the valve body 15 away from the respiratory mask 2 and against the valve body seat 14. The valve body seat 14 comprises the sealing surface 16 and a holding component 18 which is attached to the inside of the base body 10. A suitable projection prevents the holding component 18 from being moved linearly away from the respiratory mask 2. The sealing surface 16 is applied to the holding component 18. A guide pin 17 in the holding component 18 guides the valve body 15 in the two opposite directions, namely towards the valve body seat 14 and away from the valve body seat 14.

The piece 100 comprises two coupling points, namely a mask-side coupling point 21 and a device-side coupling point 22. With the help of the mask-side coupling point 21, the piece 100 can be detachably connected to the coupling point 20 of the respiratory mask 2. The device-side coupling point 22 is explained below with reference to FIG. 4. Breathing air flows in a flow direction F from the device-side coupling point 22 through the fluid connection D to the mask-side coupling point 21 and further to the interior space I. A display surface of the pressure gage 12 points towards the user of the respiratory mask 2.

In addition, FIG. 3 schematically shows an exhalation valve 25 of the respiratory mask 2. The exhalation valve 25 opens if a sufficiently high overpressure relative to the environment occurs in the interior space I. For example, the exhalation valve 25 opens at an overpressure of 4.5 mbar.

FIG. 4 shows the piece 100 and the respiratory mask 2, as also shown in FIG. 2 and FIG. 3, as well as a breathing air source 30. The breathing air source 30 is capable of providing breathing air under overpressure. The piece 100 is arranged between the breathing air source 30 and the respiratory mask 2. The piece 100 therefore functions as an intermediate piece in the sense of the disclosure.

In the situation of FIG. 4, the valve 11 is open. In the example shown, the valve 11 is opened as follows: The breathing air source 30 is connected to the piece 100. In one embodiment, an overpressure of the breathing air source 30 moves a guide element 27 in the breathing air source 30. In another embodiment, an inhaling user causes this movement.

The moved guide element 27 presses against a transmission element 28 of the piece 100. The transmission element 28 presses against the guide pin 17 and thereby opens the valve 11 against the restoring force of the compression spring 19, provided that the breathing air source 30 generates a sufficiently high pressure. If the valve 11 is open, a fluid connection is established from the breathing air source 30 to the respiratory mask 2. This fluid connection passes through the coupling points 23, 22, 21, 20 in this order.

The breathing air source 30 is or includes, for example, a compressed air breathing apparatus with a lung demand valve or a filter unit (not shown). FIG. 4 is a schematic view of the lung demand valve 30. The lung demand valve ensures that an overpressure of, for example, 3 mbar relative to the environment U occurs in the interior space I of the respiratory mask 2, but not a significantly greater overpressure. This overpressure reduces the risk of particles and other pollutants entering the interior space I if the respiratory mask 2 does not fit perfectly fluid-tight against the user's face.

The filter unit includes its own housing and a filter within the housing. The breathing air source 30 provides breathing air that is free of particles and other pollutants. Breathing air flows from the breathing air source 30 through the piece 100 to the respiratory mask 2 and from there into the interior space I.

The breathing air source 30 comprises a coupling point 23. In FIG. 4, the coupling point 23 is detachably connected to the device-side coupling point 22 of the piece 100. In the example shown, the connection is a snap-in connection or a screw connection or a bayonet connection. The mask-side coupling point 21 of the piece 100 is connected to the coupling point 20 of the respiratory mask 2. Again, the pressure gage 12 measures the pressure at the measuring position 13 in the fluid connection D, i.e. inside the base body 10, and the pressure gage 12 displays the measured pressure on a display unit. The measured pressure can be less than, greater than or equal to the ambient pressure.

The configuration shown in FIG. 4 can be used, for example, to test the tightness of the respiratory mask 2. The pressure inside the base body 10 is generated by the breathing air source 30 and/or by the inhaling user. An arrangement comprising the breathing air source 30 and the respiratory mask 2 can also be used without the piece 100, in particular for productive use. For this purpose, the coupling point 23 can be directly and detachably connected to the coupling point 20. In this situation, the guide element 27 engages with a corresponding receiving element 29 of the respiratory mask 2.

The coupling points 20 and 22 have the same geometry and dimensions, and the coupling points 23 and 21 also have the same geometry and dimensions. This allows the piece 100 to be arranged either between the breathing air source 30 and the respiratory mask 2, or the breathing air source 30 can be connected directly to the respiratory mask 2. These geometries and dimensions can, for example, be compatible with a standard for plug connectors, e.g. with the standard ESA (Standard-Einheits-Steckanschluss).

It is also possible for an arrangement according to the disclosure as just described to comprise at least one breathing air source 30, at least one respiratory mask 2, and two different pieces (intermediate pieces) 100, 100.a. The two intermediate pieces 100, 100.a are designed as just described with reference to FIG. 3 and FIG. 4. In a first embodiment, the mask-side coupling point 21 of the intermediate piece 100 differs from the mask-side coupling point 21.a of the additional intermediate piece 100.a in terms of geometry and/or in terms of at least one dimension. In a second embodiment, the device-side coupling point 22 of the intermediate piece 100 differs from the device-side coupling point 22.a of the additional intermediate piece 100.a in terms of geometry and/or in terms of at least one dimension. These two embodiments can be combined with each other. This makes it possible to use the disclosure for breathing air sources 30 with different coupling points 22 and/or for respiratory masks 2 with different coupling points 20.

FIG. 5 and FIG. 6 illustrate this schematically using an example. The arrangement according to FIG. 5 comprises a respiratory mask 2, an intermediate piece 100, and a breathing air source 30 with a coupling point 22. The intermediate piece 100 comprises a mask-side coupling point 21 and a device-side coupling point 22. The mask-side coupling point 21 can be detachably connected to the coupling point 20 of the respiratory mask 2, and the device-side coupling point 22 can be detachably connected to the coupling point 23. The arrangement according to FIG. 6 comprises the respiratory mask 2, an additional intermediate piece 100.a, and an additional breathing air source 30.a with a coupling point 23.a. The two respiratory masks 2 according to FIG. 5 and FIG. 6 are of the same construction or even identical, while the two breathing air sources 30 and 30.a have different coupling points 23 and 23.a. In the example shown, the two coupling points 23 and 23.a differ in their dimensions as follows: In the example shown, the two coupling points 23 and 23.a are circular, and the diameter d of the coupling point 23 is smaller than the diameter d.a of the coupling point 23.a. The additional intermediate piece 100.a comprises a mask-side coupling point 21 and a device-side coupling point 22.a. The mask-side coupling point 21 can be detachably connected to the coupling point 20 of the respiratory mask 2, the device-side coupling point 22.a can be detachably connected to the coupling point 23.a. In one embodiment, the coupling point 23.a can also be detachably connected to the coupling point 20.

### LIST OF REFERENCE SIGNS

| | |
|---|---|
| 2 | Respiratory mask, comprising the connection opening 20, rests on |
| | the face of a user, is tested for leaks |
| 10 | Base body of the piece 100, comprising the fluid guide unit with |
| | the fluid connection D and the coupling points 21 and 22, can be |
| | inserted into the connection opening 20 |
| 10.a | Base body of the additional piece 100.a, comprising the fluid guide |
| | unit with the fluid connection D and the coupling points 21 and |
| | 22.a, can be inserted into the connection opening 20 |
| 11 | Valve of the piece 100, can selectively close or release the fluid |
| | connection D, comprises the valve body 15 and the spring 19 |
| 11.a | Valve of the further piece 100.a, can optionally close or release the |
| | fluid connection D |
| 12 | Pressure gage of the piece 100, measures the negative pressure in |
| | the fluid connection D relative to the environment U and displays |
| | the measured pressure, is mounted on top of the base body 10 |
| 12.a | Pressure gage of the further piece 100.a, measures the negative |
| | pressure in the fluid connection D relative to the environment U |
| | and displays the measured pressure, is mounted on top of the base |
| | body 10.a |
| 13 | Measuring position at which the pressure gage 12 measures the |
| | negative pressure in the fluid connection D relative to the |
| | environment U |
| 14 | Valve body seat of the valve 11, comprises the sealing surface 16 |
| | and the holding component 18, arranged inside the base body 10 |
| 15 | Valve body of valve 11, rests on the valve body seat 14 if the valve |
| | 11 is closed |
| 16 | Sealing surface of the valve body seat 14, mounted on the holding |
| | component 18 |
| 17 | Guide pin, guides the valve body 15 |
| 18 | Holding component of the valve body seat 14, holds the sealing |
| | surface 16 |
| 19 | Spring, strives to pull or push the valve body 15 against the sealing |
| | surface 16 |
| 20 | Coupling point with a connection opening in the respiratory mask |
| | 2, can be detachably connected to the mask-side coupling point 21 |
| 21 | Mask-side coupling point of the base body 10, can be detachably |
| | connected to the coupling point 20 |
| 22 | Device-side coupling point of the base body 10, can be detachably |
| | connected to the coupling point 23 |
| 22.a | Device-side coupling point of the base body 10.a, can be |
| | detachably connected to the coupling point 23.a |
| 23 | Coupling point of the breathing air source 30, can be detachably |
| | connected to the device-side coupling point 22 |
| 23.a | Coupling point of the additional breathing air source 30.a, can be |
| | detachably connected to the device-side coupling point 22.a |
| 25 | Exhalation valve of the respiratory mask 2 |
| 27 | Guide element of the breathing air source 30, moves the |
| | transmission element 28, corresponds to the receiving element 29 |
| 28 | Transmission element of the piece 100, is moved by the guide |
| | element 27, moves the guide pin 17 |
| 29 | Receiving element of the respiratory mask 2, corresponds to the |
| | guide element 27 |
| 30 | Source of breathing air, for example a compressed air breathing |
| | apparatus or a lung demand valve or a filter unit, includes the |
| | coupling point 23 |
| 30.a | Another source of breathing air, includes the coupling point 23.a |
| 100 | Piece, comprising the respiratory mask 2, the base body 10 with |
| | the coupling points 21 and 22, the valve 11 and the pressure gage |
| | 12, acts as an intermediate piece |
| 100.a | Additional piece, comprising the respiratory mask 2, the base body |
| | 10.a with the coupling points 21 and 22.a, the valve 11.a and the |
| | pressure gage 12.a, acts as an additional intermediate piece |
| d | Diameter of the circular coupling point 23 |
| d.a | Diameter of the circular coupling point 23.a |
| D | Fluid connection between the environment and the interior space I, |
| | passes through the base body 10 |
| F | Flow direction in which breathing air flows through the fluid |
| | connection D into the interior space I |
| I | Interior space between the respiratory mask 2 and the user's face |
| U | Environment |

1.-8. (canceled)

9. An arrangement comprising:
a respiratory mask with a respiratory mask coupling point;
a breathing air source with a breathing air source coupling point,
an intermediate piece; and
a pressure sensor;
wherein the intermediate piece comprises a device-side coupling point and a mask-side coupling point and provides a fluid connection between the device-side coupling point and the mask-side coupling point;
wherein the pressure sensor is configured to measure a pressure at a measuring position;
wherein the measuring position of the pressure sensor is located in the fluid connection provided by the intermediate piece;
wherein the respiratory mask is configured to rest against a user's face;
wherein the breathing air source is configured to provide breathing air;
wherein the device-side coupling point is configured to be detachably connected to the breathing air source coupling point;
wherein the mask-side coupling point is configured to be detachably connected to the respiratory mask coupling point;
wherein the breathing air source coupling point is configured to be detachably connected to the respiratory mask coupling point;
wherein, if the device-side coupling point is connected to the breathing air source coupling point and the mask-side coupling point is connected to the respiratory mask coupling point, a first fluid connection from the breathing air source to the respiratory mask is established and the first fluid connection passes through the intermediate piece; and
wherein, if the breathing air source coupling point is connected to the respiratory mask coupling point, a second fluid connection from the breathing air source to the respiratory mask is established.

10. The arrangement according to claim 9, wherein the intermediate piece comprises a valve;
wherein the valve, in a closed state, is configured to block the fluid connection provided by the intermediate piece;
wherein the valve, in a releasing state, is configured to release the fluid connection provided by the intermediate piece; and
wherein the valve is configured such that the valve is in the releasing state if the pressure at the mask-side coupling point is lower than the pressure at the device-side coupling point by at least a predetermined pressure difference, and is otherwise in the closed state.

11. The arrangement according to claim 9, wherein the pressure sensor comprises a display unit; and
wherein the display unit is positioned so that if the mask-side coupling point is connected to the respiratory mask coupling point, the display unit points towards the respiratory mask such that a user of the respiratory mask can read the display unit.

12. The arrangement according to claim 9, further comprising:
an additional intermediate piece;
an additional breathing air source comprising an additional breathing air source coupling point; and
an additional pressure sensor;
wherein the additional intermediate piece comprises an additional device-side coupling point and an additional mask-side coupling point and provides an additional fluid connection between the additional device-side coupling point and the additional mask-side coupling point;
wherein the additional pressure sensor is configured to measure the pressure at an additional measuring position in the additional fluid connection which is provided by the additional intermediate piece;
wherein the additional breathing air source is configured to provide breathing air;
wherein the additional device-side coupling point is configured to be detachably connected to the additional breathing air source coupling point;
wherein the additional mask-side coupling point is configured to be detachably connected to the respiratory mask coupling point; and
wherein, if the additional device-side coupling point is connected to the additional breathing air source coupling point and the additional mask-side coupling point is connected to the respiratory mask coupling point, an additional first fluid connection is established from the additional breathing air source to the respiratory mask and the additional first fluid connection passes through the additional intermediate piece.

13. The arrangement according to claim 12, wherein the additional breathing air source coupling point is configured to be detachably connected to the respiratory mask coupling point; and
wherein, if the additional breathing air source coupling point is connected to the respiratory mask coupling point, an additional second fluid connection is established from the additional breathing air source to the respiratory mask.

14. A method for operating the arrangement of claim 9, the method comprising:
placing the respiratory mask on the user's face;
connecting the intermediate piece to the respiratory mask and to the breathing air source so that the first fluid connection from the breathing air source to the respiratory mask is at least temporarily established;
wherein the first fluid connection passes through the intermediate piece;
measuring, with the pressure sensor, a pressure at the measuring position;
separating the intermediate piece from the respiratory mask and from the breathing air source; and
connecting the breathing air source to the respiratory mask such that the second fluid connection from the breathing air source to the respiratory mask is at least temporarily established.

15. The method according to claim 14, the method further comprising:
connecting the intermediate piece to the breathing air source;
after the intermediate piece has been connected to the breathing air source and before connecting the intermediate piece to the breathing air source, closing the mask-side coupling point in a fluid-tight manner with a closure;
measuring, with the pressure sensor, the pressure while the closure closes the mask-side coupling point;
removing the closure; and
connecting the mask-side coupling point to the respiratory mask coupling point.

16. A method for operating the arrangement of claim 10, the method comprising:
placing the respiratory mask on the user's face;
connecting or keeping connected the intermediate piece to the respiratory mask and separating or keeping separated the intermediate piece from the breathing air source, such that a third fluid connection from an environment to the respiratory mask is at least temporarily established;
wherein the third fluid connection passes through the intermediate piece;
bringing or keeping the valve in the closed state;
measuring the pressure at the measuring position with the pressure sensor while the valve is in the closed state;
separating the intermediate piece from the respiratory mask; and
connecting the breathing air source to the respiratory mask, such that the second fluid connection is established from the breathing air source to the respiratory mask.
